(19) United States
(12) Patent Application Publication
(10) Pub. No.: US 2005/0281862 A1
(43) Pub. Date: Dec. 22, 2005

(54) Non-aspirating transitional viscoelastics for use in surgery

(76) Inventors: Mutlu Karakelle, Fot Worth, TX (US); David L. Jinkerson, Benbrook, TX (US); Michael R. Brunstedt, Cleveland, OH (US); Anilbhai S. Patel, Arlington, TX (US); Kwan Y. Chan, Fort Worth, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 11/189,669

(22) Filed: Jul. 25, 2005

Related U.S. Application Data
(62) Division of application No. 10/111,524, filed on Apr. 24, 2002, now abandoned.
(60) Provisional application No. 60/246,181, filed on Nov. 6, 2000.
(60) Provisional application No. 60/246,187, filed on Nov. 6, 2000.

(51) Int. Cl. A61K 31/728

(52) U.S. Cl. 424/427; 514/54

(57) ABSTRACT

Non-aspirating viscoelastics, compositions, and methods of use are disclosed. The non-aspirating, transitional viscoelastics possess sufficient viscosity to be useful in ophthalmic viscosurgery, but may be left in the eye with little or no resulting IOP spike. The compositions are particularly useful in cataract surgery.

### FIELD OF THE INVENTION

The present invention relates to the field of viscous and viscoelastic materials suitable for use in surgical procedures. In particular, non-aspirating viscoelastics, including transitional viscoelastics having non-shear related variable viscosities), which may be left in situ at the close of surgery are disclosed. Methods of using transitional viscoelastics in surgery, especially ophthalmic surgery are also disclosed.

### BACKGROUND OF THE INVENTION

Viscous or viscoelastic agents used in surgery may perform a number of different functions, including without limitation maintenance and support of soft tissue, tissue manipulation, lubrication, tissue protection, and adhesion prevention. It is recognized that the differing rheological properties of these agents will necessarily impact their ability to perform these functions, and, as a result, their suitability for certain surgical procedures. See, for example, U.S. Pat. No. 5,273,056.

Cataracts are opacities of the ocular lens which generally arise in the elderly. In order to improve eyesight, the cataractous lens is surgically removed and an artificial intraocular lens is inserted in its place. During these surgical procedures, viscoelastic materials are typically injected in the anterior chamber and capsular bag to prevent collapse of the anterior chamber and to protect tissue from damage resulting from physical manipulation.

A number of viscous or viscoelastic agents (hereinafter “agents”) are known for ophthalmic surgical use. For example, Viscoat® (Alcon Laboratories, Inc.) which contains sodium hyaluronate and chondroitin sulfate; Healon® and Healon® GV (Pharmacia Corp.), Amvisc® Regular and Amvisc® Plus (IOLAB), and Vitrax® (Allergan) all of which contain sodium hyaluronate; and Cellugel® (Alcon) which contains hydroxypropylnethylcellulose (HPMC) are all useful in cataract surgery. They are used by the skilled ophthalmic surgeon for several purposes, including maintenance of the anterior chamber of the eye and protection of ophthalmic tissues during surgery, particularly corneal endothelial cells, and as an aid in manipulating ophthalmic tissues.

While all of the agents described above may be used during cataract surgery, each has certain recognized advantages and disadvantages. See, U.S. Pat. No. 5,273,056. Generally, however, all such agents having sufficient viscosity and pseudoplasticity to be useful in ophthalmic surgery will, if left in the eye at the close of surgery, result in a transient increase in intraocular pressure (“IOP”) known as an “IOP spike.” (See, Obstbaum, Postoperative pressure elevation. A rational approach to its prevention and management, J. Cataract Refractive Surgery 18:1 (1992).) The pressure increase has been attributed to the agent's interference with the normal outflow of aqueous humor through the trabecular meshwork and Schlemm's canal. (See, Berson et al., Obstruction of Aqueous Outflow by Sodium Hyaluronate in Enucleated Human Eyes, Am. J. Ophthalmology, 95:668 (1983); Olivius et al., Intraocular pressure after cataract surgery with Healon®, Am. Intraocular Implant Soc. J. 11:480 (1985); Fry, Postoperative intraocular pressure rises: A comparison of Healon, Amvis, and Viscoat, J. Cataract Refractive Surgery 15:415 (1989).) IOP spikes, depending on their magnitude and duration, can cause significant and/or irreversible damage to susceptible ocular tissues, including, without limitation, the optic nerve.

Thus, the ease with which an agent can be removed from the surgical site, typically by aspiration, has traditionally been considered an important characteristic in the overall assessment of the agent's usefulness in cataract surgery. By removing the agent before the close of surgery, the surgeon hopes to minimize or avoid any significant IOP spike. Unfortunately, however, removal of agents which are relatively dispersive (as opposed to cohesive) or which adhere to the ocular tissue is often difficult and may cause additional trauma to the eye.

Exogenous dilution of the viscoelastic has been suggested to alleviate IOP spikes. See U.S. Pat. No. 4,328,803. Depending, however, on the particular viscoelastic and the surgical technique employed, IOP spike may still be a problem. More recently, it has been suggested that the administration of degradative agents to break down conventional viscous or viscoelastic agents in the eye can reduce or avoid the occurrence of IOP spikes. See, e.g., U.S. Pat. No. 5,792,103. Such an approach requires not only the administration of a second, enzymatic agent into the eye, the biocompatability of which must be assured; but also means for adequately mixing the two agents in a special apparatus.

Viscoelastics have also been promoted as drug delivery devices for pharmaceutical agents which are administered when the viscoelastics are applied during surgery. For example, U.S. Pat. No. 5,811,453 (Yanni et al.) discloses viscoelastics containing anti-inflammatory compounds and methods of using these enhanced viscoelastics in cataract surgery. While this approach may ameliorate ocular inflammation resulting from surgical trauma, such an approach still possesses the significant limitation of presenting IOP spike problems, as described above. Consequently, these enhanced viscoelastics still need to be aspirated out at the close of surgery.

There is, therefore, a need for an improved means for reducing or avoiding IOP spikes associated with the use of conventional viscous or viscoelastic agents in ophthalmic surgery, especially cataract surgery. More specifically, we conceived the need for an improved viscous or viscoelastic agent having a variable or transitional viscosity such that it will, without the addition of degradation agents, become substantially less viscous after its purpose has been served in surgery, such agents being hereinafter referred to as transitional viscoelastics. A significant amount of such a transitional viscoelastic may then be left in the eye by the surgeon to be eliminated by the body's natural processes without creating a dangerous IOP spike.

Transitional viscosities are known to occur in certain systems. In the ophthalmic field, systems are known in which a liquid forms a gel after application to the eye. For example, such gelations may be triggered by a change in pH. See, Gurney et al., “The Development and Use of In Situ Formed Gels, Triggered by pH” Biopharm. Ocul. Drug Delivery, (1993) pp. 81-90. Temperature sensitive gelation systems have also been observed for certain ethyl (hydroxyethyl) cellulose ethers (EHECs) when mixed with particular ionic surfactants at appropriate concentrations (see, Carlsson et al., “Thermal Gelation of Nonionic Cellulose Ethers and Ionic Surfactants in Water” Colloids Surf., volume 47, pages 147-65 (1990)) and for systems of pure methylethyl cellulose (U.S. Pat. No. 5,618,800 (Kabra et al.)) More recently, in U.S. Pat. No. 6,177,544, a modified collagen for ophthalmic use was disclosed, which was reported to lose viscosity upon denaturation to facilitate removal. However, no commercial embodiments of such material are believed to be available. It is also known that carrageenans can be tailored to adjust their viscosity transitions to different temperature ranges. (See, Verschueren et al. “Evaluation of various carrageenans as ophthalmic viscolysers” STP Pharma Sci, volume 6, pages 203-210 (1996), and Picullel et al., “Gelling Carrageenans,” Food Polysaccharides and Their Applications, Ed: Stephen, A.M., Marcel Dekker-New York, volume 67, pages 204-44 (1995).) Finally, gellan gum (Gelrite®) is known to form a gel on contact with specifications. Greaves et al., “Scintigraphic Assessment of an Ophthalmic Gelling Vehicle in Man and Rabbit,” Curr. Eye Res., volume 9, page 415 (1990). Gellan systems have been suggested for use as a vehicle for ophthalmic medications (Rozier et al., “Gelrite: A Novel, Ion-Activated, In Situ Gelling Polymer for Ophthalmic Vehicles. Effect on Bioavailability of Timolol,” Int. J. Pharm., volume 57, page 163 (1989)), and one gellan system is currently being marketed with timolol, a beta blocker, as a glaucoma medication.

The use of a non-collagen based transitional viscosity viscoelastic agent as an effective surgical tool, however, especially in ophthalmic surgery, has neither been disclosed or suggested in the art. To be most effective for use as an ophthalmic surgical tool, the agent, in addition to having the desired initial and transitional viscosities over the prescribed temperature range, would preferably meet the following requirements: physiologically acceptable osmolality and pH; relatively short viscosity transition time; clear (without turbidity); biocompatible; and sterilizable. The transitional viscoelastics of the present invention are believed to satisfy these requirements.

### SUMMARY OF THE INVENTION

The present invention is directed to improved viscous or viscoelastic agents for use in surgical procedures, especially ophthalmic surgical procedures. More specifically, the present invention is directed to any such agent with the desired initial viscosity that yields an acceptable IOP spike profile in the IOP Spike Model described below. The improved agents of the present invention include transitional viscous or viscoelastic polymeric agents suitable for use in ophthalmic surgery. As used herein, the term “transitional viscoelastic” means such an agent which maintains high viscosity during the surgical procedure, but rapidly loses viscosity after the close of surgery so as to reduce or avoid the occurrence of dangerous IOP spikes, and to reduce or obviate the need for active removal of the viscoelastic at the end of the surgical procedure.

Appreciating that the surface temperature of the eye tissues during surgery will approximate room temperature, or about 25° C. or less, we have discovered agents that will maintain suitable viscosity at that temperature, but will rapidly lose viscosity at a slightly higher temperature (i.e., body temperature, approximately 37° C.). The loss of viscosity, which occurs without the exogenous addition of a degradation agent, results primarily from the warming of the eye back to body temperature after the surgery is complete.

The stability of the transitional viscoelastics of the present invention is a particularly important feature of the present invention. If the agent undergoes substantial hydrolysis, oxidation or other degradation prior to use, the agent may lose its viscous properties and yield a non-useful or less useful viscoelastic. The preferred transitional viscoelastic compositions of the present invention are substantially stable, exhibiting less than 1% degradation for up to six months at storage temperatures. These compositions will yield little or no IOP spike (as defined below) when used and allowed to remain in the eye after routine cataract surgery.

Substances suitable for use as transitional viscoelastics include, without limitation, hydrophobically modified polysaccharides or mucopolysacrharides such as hyaluronic acid and its salts (HA) (with or without surfactants); dialyzed polyampholytes or dialyzed mixtures of oppositely charged polyelectrolytes; polysaccharides or mucopolysaccharides such as HA with cationic hydrophilic polymers; polysaccharides and hydrophilic synthetic polymers with temperature dependent conformational transitions; and combinations thereof. Preferred are hydrophobically modified polysaccharides or mucopolysaccharides. Most preferred are hydrophobically modified HAs, especially HA-amides.

### DETAILED DESCRIPTION OF THE DRAWINGS

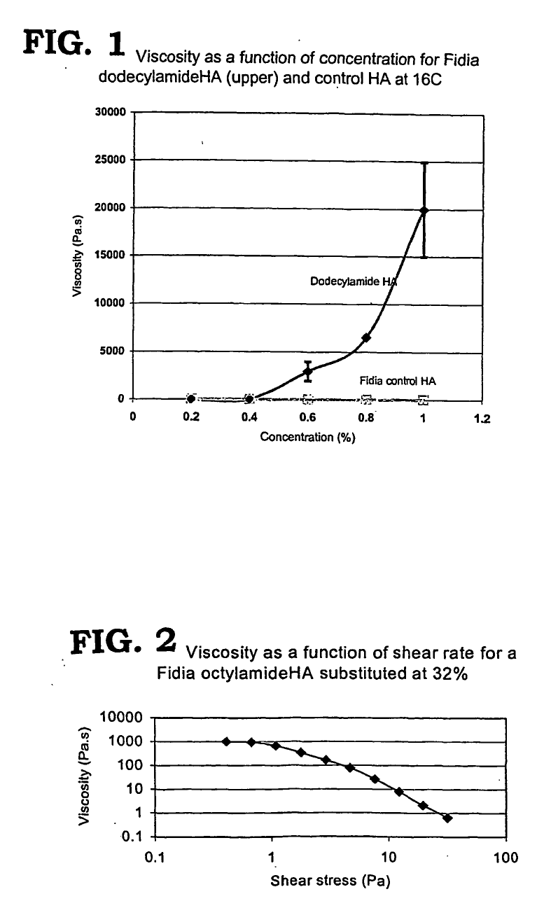

FIG. 1 is a graph depicting viscosity as a function of concentration for dodecylamide HA of the present invention and control HA.

FIG. 2 is a graph depicting viscosity as a function of shear rate for octylamide HA of the present invention.

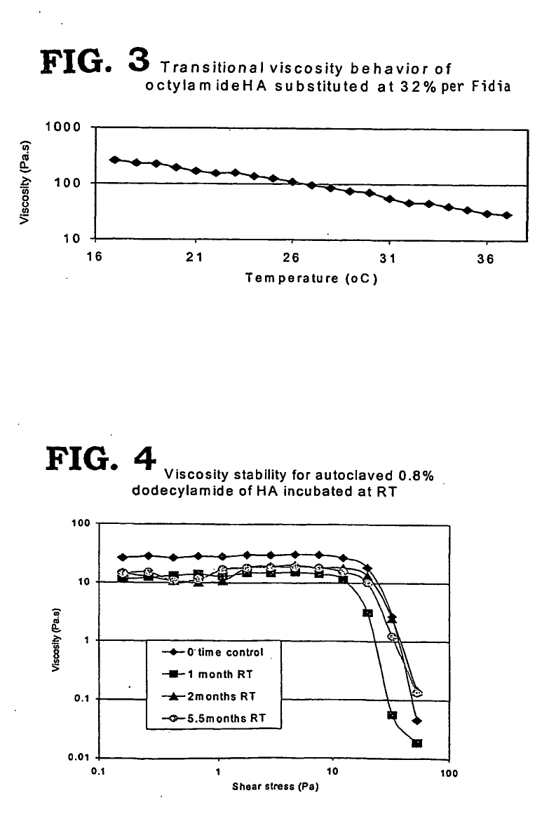

FIG. 3 is a graph depicting the transitional viscosity of octylamide HA of the present invention.

FIG. 4 is a graph depicting the viscosity stability of autoclaved dodecylamide HA of the present invention.

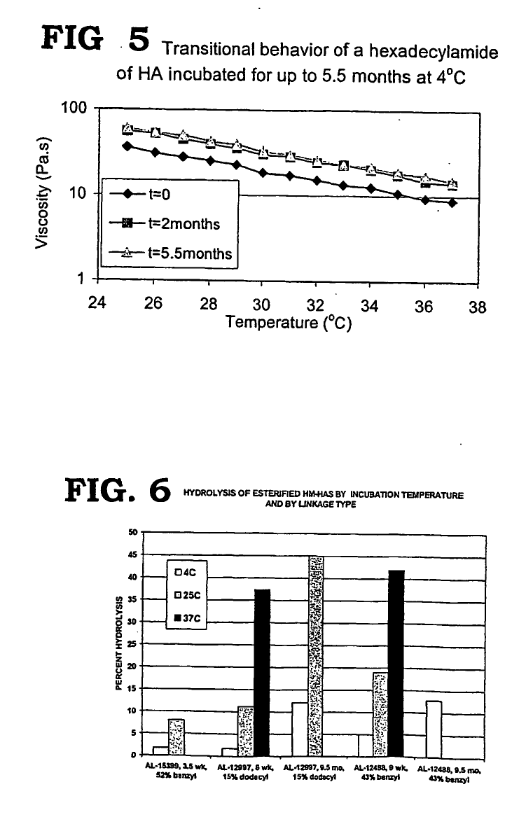

FIG. 5 is a graph depicting the stability of the transitional behavior of hexadecylamide HA of the present invention.

FIG. 6 is a graph depicting the rate of hydrolysis of esterified HAs of the present invention.

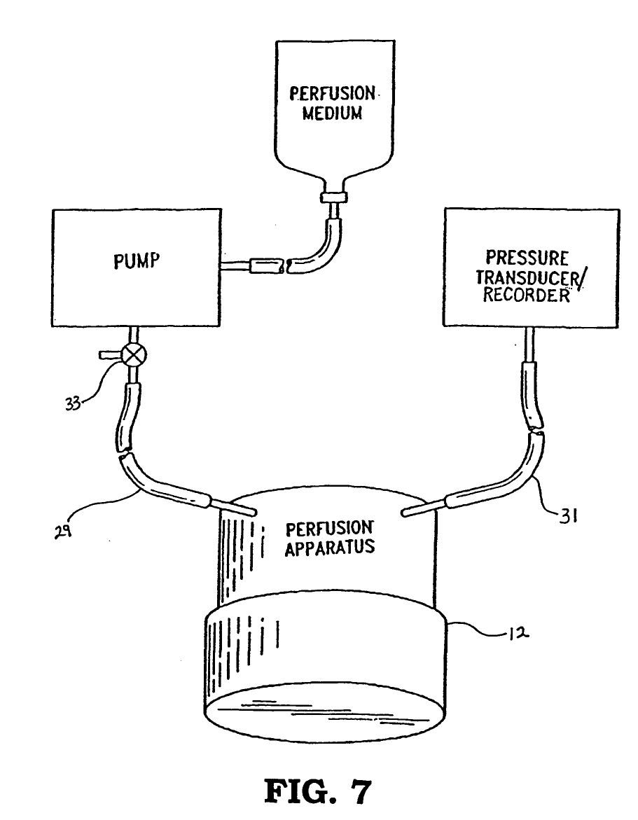

FIG. 7 is a diagrammatic representation of the IOP Spike Model of the present invention.

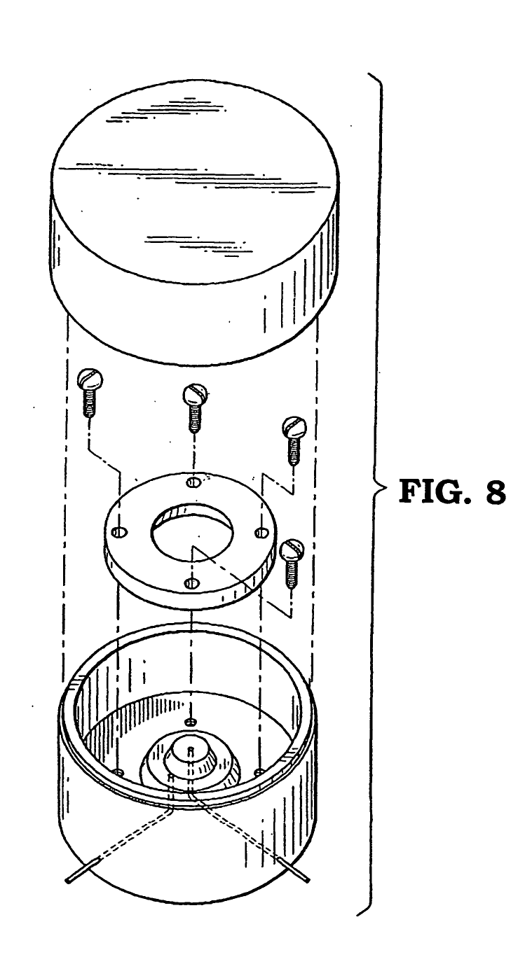

FIG. 8 is an exploded elevation of a perfusion apparatus of the present invention.

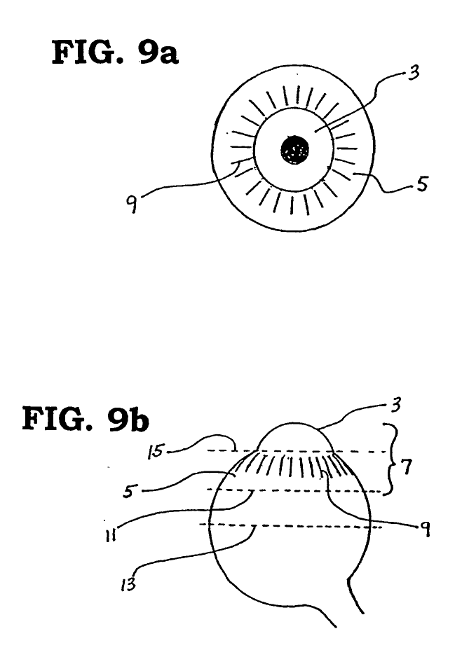

FIG. 9a is a top plan view of an eye for use in a perfusion apparatus.

FIG. 9b is a side view of the eye of FIG. 9a.

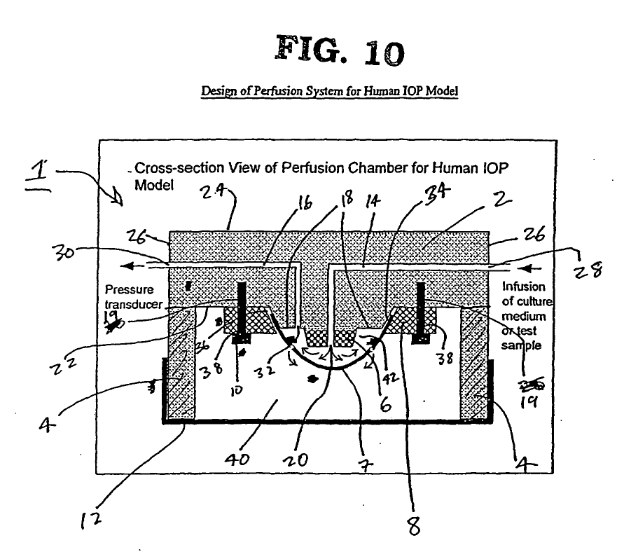

FIG. 10 is a cross sectional view of the perfusion apparatus of the present invention FIG. 8 with the inclusion of an anterior segment.

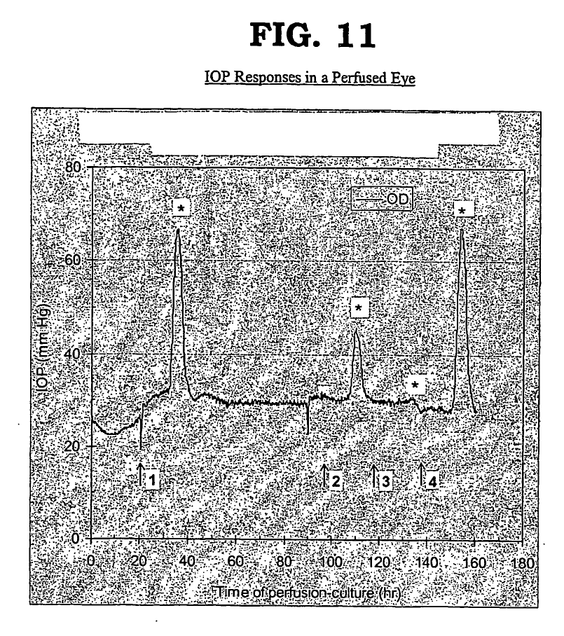

FIG. 11 is a graph depicting the effects of traditional viscoelastics and a transitional viscoelastic of the present invention on IOP using the IOP Spike Model of the present invention.

### DETAILED DESCRIPTION OF THE PRESENT INVENTION

The present invention is directed to viscoelastic materials, and especially to transitional viscoelastic materials, compositions and methods of use. The primary use of the transitional viscoelastics is in surgical applications where the transitional viscoelastic is applied during surgery in its more viscous state and, following surgery, loses substantial viscosity in situ. A preferred use of the transitional viscoelastics is in cataract surgery, where the viscoelastic is instilled i) in the anterior chamber of the eye to maintain the dome and protect the exposed tissues; and/or ii) in the posterior chamber to inflate the capsular bag. Following surgery, the viscoelastic remaining in the eye is heated by the body to ambient body temperature, loses its viscosity, and is more readily removed (than non-transitional viscoelastics) by the eye's processes. The major advantage of this preferred use is the avoidance of the IOP spike that may occur with other systems. Thus, another advantage of this use is that it allows the surgeon the traditional advantages of a viscoelastic without the disadvantage of having to thoroughly aspirate the viscoelastic out of the surgical site following completion of the surgery. As stated above, such aspiration is time consuming and presents additional risk to the patient.

The transitional viscoelastics of the present invention typically exhibit a viscosity loss of 70% or more, without substantial hydrolysis, when such materials undergo a temperature change of from about room temperature or surgical temperature (approximately 17-26° C.) to about body temperature (approximately 35-38° C.).

As stated above, the preferred transitional viscoelastics of the present invention will be substantially stable. As used herein, “substantially stable” refers to viscoelastics that only lose 1% or less of their hydrophobic side chains by hydrolysis, oxidation or other degradation, when such viscoelastics are stored refrigeration temperatures of approximately 4° C. for up to 6 months.

The transitional property of the present invention viscoelastics is preferably reversible. The reversible viscosity property of the preferred embodiments allows the transitional viscoelastics to be heated prior to use, e.g., heat sterilization, and then recooled for surgical application.

Additional preferred properties of the transitional viscoelastics of the present invention include: (1) a transition time of less than about two hours post surgery; (2) optically clear gels with little or no turbidity; (3) safe adherence to ocular tissue (i.e., capability of providing a protective coating to delicate tissues); and (4) biocompatibility.

As stated above, when used in cataract surgical procedures, the most important feature of the transitional viscoelastics of the present invention is that they will cause little or no IOP spike following such surgery. For purposes of the present invention, a transitional viscoelastic material will be deemed to exhibit “little or no IOP spike” if 0.5 ml of a 10% solution (i.e., the actual product composition diluted to 10% of its original concentration with a buffered isotonic salt solution) yields an IOP spike which is not more than an average of about 10 mm Hg above the baseline IOP in a validated IOP spike model (the “IOP Spike Model”) as described below.

While bound by no theories, we postulate that the transitional viscoelastic character of the compositions of the present invention may be attributable to physical associations between relatively low molecular weight molecules resulting in a viscosity beyond what would be expected from such low molecular weight molecules at a given concentration. Typically, the transitional viscoelastics of the present invention are modified viscoelastics wherein hydrophobic side chains have been covalently linked to the viscoelastic compounds. The unmodified viscoelastics may be substituted in varying degrees with various moieties to yield transitional viscoelastics of the present invention. For example, all of the appropriate side chains (e.g., for esters and amide transitional viscoelastics described further below, the carboxylate side chains) of the viscoelastics could be substituted (i.e., 100% substitution) or only a fraction of the side chains, e.g., 15% substitution. In general, transitional viscoelastics will be derived from known viscoelastics and modified to exhibit the properties discussed above. Examples of commercially available viscoelastics useful in the preparation of transitional viscoelastics include salts of hyaluronic acid, (e.g., sodium hyaluronate (HA)), chondroitin sulfate (CS) and hydroxypropylmethylcellulose (HPMC) and a combination of HA and CS. Other viscoelastics useful in preparing transitional viscoelastics include dialyzed polyampholytes, such as, carboxymethylcellulose.

The transitional viscoelastics may be composed of viscoelastic polymers of varying molecular weight. Generally, the average molecular weight of the non-modified polymer will range from 50,000 to 1,000,000 daltons. For HA-based transitional viscoelastics, the average molecular weight of the non-modified HA polymer backbone will preferably range from about 120,000 to about 400,000 daltons (weight average molecular weight) and from about 50,000 to about 350,000 daltons (number average molecular weight). These preferred non-modified HAs at conventional concentrations will exhibit insufficient viscosity for the preferred surgical purposes. The molecular weight of viscoelastics may be estimated by the method of gel permeation chromatography (GPC), also referred to as size exclusion chromatography (SEC), with detection by light scattering or against standards of known molecular weight using refractive index detection. Molecular weight typically affects the degree of viscosity of these known viscoelastics. All of the molecular weights pertaining to the transitional viscoelastics described herein, unless otherwise noted, are the number average molecular weights of the unmodified viscoelastic polymer prior to modification to yield a transitional viscoelastic.

The HAs (free acid and salt form) may be modified to exhibit the above properties and hence be useful as transitional viscoelastics of the present invention. For example, dodecyl moieties may be covalently linked to the backbone carboxylic acid groups of the HAs to form dodecyl esters thereof. As used herein, HAs modified by esterification of their side chains with various moieties are referred to as “HA-esters.” Examples of esters that may be substituted on the carboxylate groups of HA include, but are not limited to, alkyl groups such as methyl, ethyl, propyl, isopropyl, butyl, sec-butyl, tert-butyl, pentyl, neopentyl, hexyl, heptyl, octyl, nonyl, decyl, undecyl, dodecyl, tridecyl, tetradecyl, pentadecyl, hexadecyl, heptadecyl, octadecyl, nonadecyl, or any other alkyl groups containing up to 30 carbons; cycloalkyl groups, e.g. cyclohexyl; and aryl groups, e.g. phenyl; and any isomers of the foregoing groups. Such substituents may optionally be further substituted and may optionally contain hetero atoms selected from the group consisting of O, N and S. Such HA-esters are available from Fidia Advanced Biopolymers (Abano Terme, Italy), or may also be synthesized by methods known in the art, e.g., U.S. Pat. Nos. 5,466,461; 5,616,568; and 5,652,347; the contents of which are by this reference incorporated herein. The degree and type of such substitution will affect the low-shear or apparent viscosity and low-shear cohesion, as well as the elevated temperature viscosity and cohesion. Preferred are hydrophobic substituents.

Since there are several factors influencing the rheological properties of compositions of the present invention (e.g., type and degree of substitution and average molecular weight and concentration of the viscoelastic polymer (unmodified), various compositions of varying parameters may yield similar rheological properties. For example, a 0.88% w/v solution of a 200 kDal HA that is 14% substituted with dodecylated carboxyl groups, a 1.2% w/v solution of a 200 kDal HA that is 11% substituted with dodecylated carboxyl groups, and a 2.55% w/v solution of a 200 kDal HA that is 4% substituted with hexadecylated carboxyl groups all display similar viscosity and transitional behaviors. The transitional viscoelastics of the present invention may thus be characterized by a “Viscosity Factor,” which is determined by the following formula:

$$\begin{matrix}concentration \\ ( w / v \% )\end{matrix} \times \begin{matrix}molecular weight \\ ( \begin{matrix}unmodified polymer \\ in kilodaltons\end{matrix} )\end{matrix} \times \begin{matrix}percent \\ substitution\end{matrix} = \begin{matrix}Viscosity \\ Factor\end{matrix}$$

Transitional viscoelastics of the present invention will have Viscosity Factors ranging from about 200 to about 50,000. Preferred transitional viscoelastics of the present invention will have Viscosity Factors ranging from about 1000 to about 20,000. Most preferred are those transitional viscoelastics having Viscosity Factors from about 2000 to about 10,000.

Just as one skilled in the art will appreciate that compositions of varying parameters may yield similar rheological properties (see preceding paragraph), it will also be appreciated that because of the interplay of the parameters, compositions with the same or similar Viscosity Factor may have significantly different rheological properties. The Viscosity Factor is only a general indicator of the suitability of a viscoelastic composition for the presently contemplated purposes. Those skilled in the art will further appreciate that by modifying one or more of the parameters, optimal rheological properties for a given purpose may be achieved.

Preferred modified hyaluronates include the partial amide modification of the carboxylate groups of HA with alkyl or aryl groups to form alkyl or aryl amides of HA. As used herein, such molecules are referred to as “HA-amides.” Examples of amides that may be substituted on the carboxylate groups of HA include, but are not limited to, alkyl groups such as methyl, ethyl, propyl, isopropyl, butyl, sec-butyl, tert-butyl, pentyl, neopentyl, hexyl, heptyl, octyl, nonyl, decyl undecyl, dodecyl, tridecyl, tetradecyl, pentadecyl, hexadecyl, heptadecyl, octadecyl, nonadecyl, or any other alkyl groups containing up to 30 carbons; cycloalkyl groups, e.g. cyclohexyl; and aryl groups, e.g. phenyl; and any isomers of the foregoing groups. Such substituents may optionally be further substituted and may optionally contain hetero selected from the group consisting of O, N and S. The most preferred amide substituting group is dodecyl.

The degree of substitution may also vary. In general, the substitution will be from about 2 to 60%. The preferred substitution level will be from about 5 to 40%. Preferred amide substituting groups are octyl, dodecyl, and hexadecyl; dodecyl being the most preferred. For the octylamide HAs, the preferred variables are: substitution level of 30 to 40%; polymeric concentration of I to 3% by weight; and molecular weight of the unmodified HA of 200 to 350 kilodaltons (“kDal”) (weight average) or 120 to 230 kDal (number average). For the dodecylamide HAs, the preferred variables will be: substitution level of 5 to 32%, more preferably 15 to 25%, and most preferably 10-20%; polymeric concentration of 0.35 to 1.2% by weight; and molecular weight of the unmodified HA of 200 to 350 kDal (weight average) or 120 to 230 kDal (number average). Alternatively, a lower molecular weight unmodified HA may be used. Preferred parameters for such lower molecular weight transitional viscoelastic material would be: unmodified HA with a molecular weight of 50 to 150 kDal (weight average), amide (preferably dodecyl) substitution level of 25 to 40%, and a polymeric concentration of 0.5 to 2% (wt./v.). For the hexadecylamide HAs, the preferred variable will be: substitution level of 5 to 15%; polymeric concentration of 0.3 to 0.8% by weight; and molecular weight of the unmodified HA of 200 to 350 kDal (weight average) or 120 to 230 kDal (number average). Substitution levels may be determined by NMR as described in Example 11. In most instances, the substitution levels specified in the examples herein were provided by the supplier of the HA-amides, Fidia Advanced Biopolymers.

The transitional viscoelastic compositions of the present invention will generally have sufficient zero shear viscosity to be useful in viscosurgical procedures. Typically such zero shear viscosities will be at least 1 Pa-s at 25° C. Preferred are compositions exhibiting zero shear viscosities from about 5 to 10,000 Pa-s at 25° C. Most preferred are those exhibiting zero shear viscosities from about 40 to 1000 Pa-s at 25° C.

The HA-amides may be obtained commercially from Fidia Advanced Biopolymers (Abano Terme, Italy), may be synthesized by methods described by Danishefsky and Siskovic in “Conversion of Carboxyl Groups of Mucopolysaccharides in Amides of Amino Acid Esters,” Carbohydrate Res. Volume 16, pages 199-205 (1971), Bulpitt and Aeschlimann “New strategy for chemical modification of hyaluronic acid: Preparation of functionalized derivatives and their use in the formation of novel biocompatible hydrogels,” Biomed. Mater. Res. Volume 47, pages 152-169 (1999), or may be synthesized by other methods. WO 00/01733 (Bellini et al.), which discloses amides of HA and a process for their preparation, is by this reference incorporated herein. This reference generally discloses the use of such amides as vehicles for drug delivery for use in viscoelastic surgery or in ophthalmic surgery, but does not disclose or suggest the novel compositions and methods of the present invention.

Other transitional viscoelastics of the present invention include modified HAs wherein the hydrophobic group is linked to the HA structure through the hydroxyl moieties, the N-acetamide moieties, or the carboxyl groups, and have been converted to form hydrophobic amine (“HA-amines”), ether (“HA-ethers”), thioether (“HA-thioethers”) and alkyl (“HA-alkyls”) side chains. Examples of such transitional viscoelastics include HA alkyl ethers, HA alkylamines, HA alkyl thioethers, HA alkylcarbamates HA alkylthiocarbamates, HA alkylthioureas, and HA alkylureas, in which the alkyl group can be methyl, ethyl, propyl, isopropyl, butyl, sec-butyl, tert-butyl, pentyl, neopentyl, hexyl, heptyl, octyl nonyl, decyl, undecyl, dodecyl, tridecyl, tetradecyl, pentadecyl, hexadecyl, heptadecyl, octadecyl, nonadecyl, or any-other alkyl group containing up to 30 carbons; and any isomer of the alkyl group, including cycloalkyl and aryl isomers. Such transitional viscoelastics may, for example, be prepared by methods disclosed in March, Advanced Organic Chemistry—Reactions, Mechanisms, and Structure, John Wiley & Sons: New York, 4 Edition, 1992.

Chondroitin sulfate (CS) of varying molecular weights may be modified similarly to the HAs, described above, in order to yield transitional viscoelastics of the present invention. For example, the carboxylate groups may be amidated in analogous fashion as described above with HA. Additionally, the hydroxyl or N-acetamide moieties of chondroitin sulfates may be converted into hydrophobic amines, ethers, thioethers, carbamates, thiocarbamates, ureas, and thioureas in the same manner as described above for HA using the same alkyl, cycloalkyl and aryl substituents in order to yield transitional viscoelastics of the present invention. Examples of such transitional viscoelastics include CS alkyl ethers, CS alkylamines, CS alkyl thioethers, CS alkylcarbamates, CS alkylthiocarbamates, CS alkylthioureas, and CS alkylureas, in which the alkyl group can be methyl, ethyl, propyl, isopropyl, butyl, sec-butyl, tert-butyl, pentyl, neopentyl, hexyl, heptyl, octyl, nonyl, decyl, undecyl, dodecyl, tridecyl, tetradecyl, pentadecyl, hexadecyl, heptadecyl, octadecyl, nonadecyl, or any other alkyl group containing up to 30 carbons; and any structural isomers of such group, including cycloalkyl and aryl isomers. Such transitional viscoelastics may be prepared by methods disclosed in March, Advanced Organic Chemistry—Reactions, Mechanisms, and Structure, John Wiley & Sons: New York, 4th Edition, 1992.

The following Examples 1-6 are examples of preferred compositions of the present invention:

### EXAMPLE 1

| | Amount |
|---|---|
| Ingredient | (% w/w) |
| 10% dodecylamide substituted HA (200 kDal) sodium salt | 0.45 |
| Dibasic Sodium Phosphate (Anhydrous) | 0.056 |
| Monobasic Sodium Phosphate (Monohydrate) | 0.004 |
| Sodium Chloride | 0.84 |
| Hydrochloric Acid/Sodium Hydroxide | QS pH to 7.4 |
| Water | QS |

### EXAMPLE 2

| Ingredient | Amount (% w/w) |
|---|---|
| 10% substituted dodecylamide-HA (200 kDal) | 0.8 |
| or 8% substituted hexadecylamide-HA (200 kDal) | |
| Dibasic Sodium Phosphate (Anhydrous) | 0.056 |
| Monobasic Sodium Phosphate (Monohydrate) | 0.004 |
| Sodium Chloride | 0.84 |
| Hydrochloric Acid/Sodium Hydroxide | QS to pH to 7.4 |
| Water | QS |

### EXAMPLE 3

| Ingredient | Amount (% w/w) |
|---|---|
| 10% substituted dodecylamide-HA, sodium | 0.2, 0.4, 0.6, 0.8 or |
| salt (200 kDal) | 1.0% |
| Dibasic Sodium Phosphate (Anhydrous) | 0.056 |
| Monobasic Sodium Phosphate (Monohydrate) | 0.004 |
| Sodium Chloride | 0.84 |
| Hydrochloric Acid/Sodium Hydroxide | to adjust pH to 7.4 |
| Water | QS |

### EXAMPLE 4

| Ingredient | Amount (% w/w) |
|---|---|
| 32% substituted Octylamide-HA, sodium salt | 1.0 |
| (200 kDal) | |
| Dibasic Sodium Phosphate (Anhydrous) | 0.056 |
| Monobasic Sodium Phosphate | 0.004 |
| (Monohydrate) | |
| Sodium Chloride | 0.84 |
| Hydrochloric Acid/Sodium Hydroxide | QS to pH to 7.4 |
| Water | QS |

### EXAMPLE 5

| Ingredient | Amount (% w/w) |
|---|---|
| 10% substituted dodecylamide-HA, sodium | 1.0 |
| salt (200 kDal) | |
| Dibasic Sodium Phosphate (Anhydrous) | 0.056 |
| Monobasic Sodium Phosphate | 0.004 |
| (Monohydrate) | |
| Sodium Chloride | 0.84 |
| Hydrochloric Acid/Sodium Hydroxide | QS to pH 7.4 |
| Water | QS |

### EXAMPLE 6

| Ingredient | Amount (% w/w) |
|---|---|
| 10% dodecylamide substituted HA sodium | 0.33, 0.37, 0.40, 0.44, 0.5, |
| salt | 0.6, 0.7 or 1 |
| Dibasic Sodium Phosphate (Anhydrous) | 0.2 |
| Monobasic Sodium Phosphate | 0.045 |
| (Monohydrate) | |
| Sodium Chloride | 0.7 |
| Hydrochloric Acid/Sodium Hydroxide | QS pH to 7.4 |
| Water | QS |

The following Examples 7-18 illustrate the rheological properties of compositions of the present invention.

### EXAMPLE 7

The rheological properties of HA-amide compositions of the present invention were compared with an analogous non-transitional HA composition through several concentrations. The 10% substituted dodecylamide-HA formulations of Example 3 (0.2%, 0.4%, 0.6%, 0.8% and 1.0% w/v) and control compositions containing the precursor non-modified HA at 0.2, 0.4, 0.6, 0.8 and 1.0%, were prepared to the according to the following procedure. Containers containing the various formulations were capped and heated for 4 days at 50° C., swirling occasionally to ensure that all the dodecylamide-HA or HA powder was immersed in the buffered liquid and that complete dissolution occurred. Each solution was then transferred to separate 5 cc syringes, capped, and centrifuged at 2500 RPM to remove bubbles. The loaded syringes were then each configured with an empty 5 cc syringe via a Dual-hub assembly. The syringes were cooled for one hour and mixed using 50 passes on their respective assemblies.

In order to perform rheological analyses, samples were aspirated onto a Bohlin CS-10 (Constant Stress Rheometer) sample plate through 27 gauge needles, with the instrument set to viscometry mode. Sample viscosities were measured versus shear rate, using a shear stress of 1.09 to 52.89 Pa at 16° C. As close to a full shear-rate sweep as was feasible was used, taking into consideration possible sample loss due to centripetal forces at the high shear rate end. For each sample, the transitional behavior (viscosity versus temperature) was then characterized, using a temperature range of 10° C. to 50° C. and a heating rate of 2.5° C./min. Each sample was processed using identical parameters. The summary results are contained in Table 1.

TABLE 1
Viscosity of 10% Substituted Dodecylamide-HA
versus control HA at 16° C.

| | Viscosity at 1.36 Pa Shear, Pa-s | |
|---|---|---|
| Concentration (% w/w) | Control HA | 10% Dodecylamide of HA |
| 0.2 | 0.0209 | 0.0300 |
| 0.4 | 0.0229 | 49.2 |
| 0.6 | 0.0262 | 2960 |
| 0.8 | 0.0300 | 6580 |
| 1.0 | 0.0330 | 19,900 |

As shown in Table 1, the dodecylamide-HA compositions were much more viscous than the unmodified HA compositions. Also, an increase in the low shear viscosity through about 5 orders of magnitude was correlated with increasing dodecylamide-HA concentration (i.e., from 0.2% to 1.0% w/v).

### EXAMPLE 8

The composition of Example 4 was rheologically evaluated. The composition was prepared in a method similar to that disclosed in Example 6.

This sample was processed rheologically using the Bohlin CS-10 controlled stress rheometer for viscosity versus shear-rate using the same procedures as described in Example 6, with a shear stress range of 0.41 to 32.58 Pa. The results are contained in Table 2.

TABLE 2
Viscosity versus Shear Stress at 16° C.
for 32% substituted Octylamide-HA

| Shear Stress (Pa) | Viscosity (Pa-s) |
|---|---|
| 0.416 | 995 |
| 0.676 | 961 |
| 1.09 | 670 |
| 1.78 | 342 |
| 2.89 | 166 |
| 4.69 | 79.0 |
| 7.61 | 27.0 |
| 12.36 | 7.88 |
| 20.07 | 2.12 |
| 32.58 | 0.65 |

As shown in Table 2, the 1% solution demonstrated a low shear viscosity of about 1000 Pa-s.

In an analogous experiment, the viscosity of the composition was tested through a temperature range of 17° C. to 37° C., with a shear stress of 1.781 Pa. The results are reported in Table 3.

TABLE 3
Viscosity versus Temperature for 1%
solution of 32% substituted Octylamide-HA

| Temperature (° C.) | Viscosity (Pa-s) |
|---|---|
| 17 | 261 |
| 22 | 155 |
| 27 | 97.0 |
| 32 | 47.3 |
| 37 | 29.5 |

As shown in Table 3, and illustrated in FIG. 3, the transitional viscosity loss behavior of this material correlates linearly with increasing temperature. The total viscosity loss over the temperature range was about 88%.

### EXAMPLE 9

The storage stability of preferred compositions of the present invention, as a measure of retained viscosity, was observed in the following experiment. The dodecylamide-HA of Example 2 was incubated at 4° C. and room temperature (“RT,” i.e., 21-23° C.) through 5.5 months. At a given time point, an aliquot of each composition was taken and rheological analysis (the shear stress range for this example was from 0.16 to 52.89 Pa.) of the sample was performed. The results are listed in Table 4 and illustrated in FIG. 4.

TABLE 4
Viscosity Stability of 0.8% solution of 10% substituted Dodecylamide-HA

| Time (months) | Viscosity at 16° C. and with a 7.61 Pa |
|---|---|
| at RT | Shear Stress (Pa-s) |
| 0 | 30.0 |
| 1 | 14.2 |
| 2 | 18.1 |
| 5.5 | 18.1 |

As shown in Table 4, dodecylamide-HA compositions experienced an initial shift of 16 Pa-s at low shear conditions in viscosity between the zero time control and 1 month incubation time point. However, the viscosity was stable from the 1 month incubation time point to the 5.5 month incubation time point.

The transitional stability of hexadecylamide-HA compositions stored at 4° C. through 5.5 months was also tested. The results are disclosed in Table 5.

TABLE 5
Viscosity versus Temperature Stability Analysis of 8% substituted
Hexadecylamide-HA Stored in Phosphate Buffered Saline at 4° C.

| | Viscosity at Shear Stress of 2.89 Pa (Pa-s) | | |
|---|---|---|---|
| Temperature | Storage Time, | Storage Time, | Storage Time, |
| (° C.) | 0 months | 2 months | 5.5 months |
| 25 | 36.2 | 55.5 | 59.8 |
| 28 | 25.3 | 39.8 | 42.6 |
| 31 | 17.3 | 28.4 | 29.7 |
| 34 | 12.6 | 20.3 | 21.3 |
| 37 | 8.8 | 14.0 | 14.8 |

As shown in Table 5 and FIG. 5, the viscosity of all compositions transitioned through the temperature change from 25° C. to 37° C., losing between 70% and 80% of their viscosity at a shear stress of 2.89 Pa. This compares to a viscosity loss of only about 35% for hyaluronic acid solutions of similar starting viscosities.

### EXAMPLE 10

The stability of a dodecylamide-HA composition of the present invention was observed with the following experiment. The 1% w/v dodecylamide-HA composition of Example 7 was incubated at 4° C., room temperature (21-23° C.) and 37° C. through 6 months. At the appropriate time, an aliquot of the composition was taken and the chemical stability of the dodecylamide-HA was analyzed using capillary gas chromatography (GC). GC was performed on the Hewlett Packard 5890A GC system equipped with a flame ionization detector (FID), using the following parameters:

| GC Parameter | Value | |
|---|---|---|
| Injection port temperature | 250° C. | |
| Initial oven temperature | 60° C. | |
| Oven temperature program | 60° C. to 300° C. at 10/min. | |
| | Hold at 300° C. for 5 min. | |
| Helium, flow rates | Split | 100 mL min |
| | Septum purge | 0.3 mL/min |
| | Column | 1.2 mL/min |
| | Make up (FID) | 30 mL/min |

| | |
|---|---|
| Compressed air (FID) | 395 mL/min |
| Hydrogen (FID) | 23.5 mL/min |

The column used was a DB5 fused silica capillary column (30 meters in length with an inner diameter of 0.32 mm and a film thickness of 1.0 mm) from J&W Scientific, (Folsom, Calif.).

After a given incubation time, samples were mixed with one weight equivalent of a mixture of two parts ethyl acetate to one part ethanol (also by weight) and incubated at 50° C. for one hour. To this mixture was added three more parts by weight of the ethyl acetate-ethanol mixture. This second addition caused precipitation of the polysaccharide, which is centrifuged down, and the supernatant was analyzed by GC for the presence of the breakdown hydrophobic group, dodecylamine. If 100% hydrolysis of the side chains of the dodecylamine-HA occurred, full precipitation of the 10% substituted dodecylamine-HA, would result in 42 ppm dodecylamine in the supernatant. The test results showed that less than 1 ppm of dodecylanine was present in the supernatants of the processed composition samples, through the various temperature and time incubations. These results indicated that the amide linkage of the transitional viscoelastic was very stable in the buffered composition.

### EXAMPLE 11

### NMR Analysis for Determination of Hydrophobic Group Substitution Level of Hydrophobically-modified Hyaluronic Acid (HM-HA) Compounds

Into 4 mL glass vials, was added 3-5 mg of a HM-HA material. Into the same vial was added 0.8 mL of water and the vial then agitated on a vortex mixer for 5 to 10 seconds. The sample vial was placed into an oven set at 50° C. and heated overnight (15-20 hours) to dissolve. The next day a hyaluronate lyase (600-900 units/sealed ampule, Cat. No. H-1136, Sigma Chemical Co.) enzyme solution was prepared by snapping open the sealed ampule and adding 0.8 mL of water to the ampule containing approximately 900 units of enzyme (1 unit/uL). To the vial was then added 100 uL (0.1 mL) of the enzyme solution. The vial was capped and placed into a 37° C. oven overnight (15-20 hrs).

The next day, the vial was removed from the oven and 100 uL (0.1 mL) of deuterium oxide (99.6% atom-% D, Cat. No. 42,345-9 Aldrich Chemical Co.) was added to the vial. After mixing, the solution was transferred into a NMR tube using a glass disposable transfer pipette. The sample was then analyzed to obtain a proton NMR spectrum on a 600 MHz Bruker NMR instrument capable of operation in a moisture suppression mode with accurate integration of peak signals.

Using the NMR spectrum of the enzyme-treated HM-HA sample, the hydrophobic substitution level was calculated from integration values by adding together the integral values for the 3 signals indicative of the hydrophobic residue from 0.8 to 1.3 ppm versus the 2 to 4 signals at 2.0-2.1 ppm for the N-acetylmethyl group.

The three signals between 0.8-1.3 ppm originate from the hydrogen atoms bonded to C₂ to Cₙ carbons in the hydrophobic group, which contains n carbon atoms. The hyaluronate lyase enzyme has no interfering signals in the hydrophobic group region or the N-acetylmethyl signal region. Since the N-acetylmethyl group is on every repeat unit in the HA structure, the hydrophobic substitution level may be calculated from the ratio of integral values for the hydrophobic group to that of the N-acetylmethyl group. Therefore, the calculation of the degree of substitution of a HM-HA substance with a straight chain normal alkyl group [—CH₂)ₙ₋₁CH₃] as its hydrophobic substitutent with n carbons can be given by the equation:

$$\begin{matrix}\% hydrophobic \\ substitution\end{matrix} = \frac{[ Integrals 0.8 - 1.5 ppm / ( 2 ( n - 1 ) + 1 ) ]}{[ Integrals 2.0 - 2.1 ppm / 3 ]} \times 100$$

### EXAMPLE 12

The following example demonstrates the lesser stability of less preferred viscoelastic agents of the present invention. Compositions analogous to those of Examples 1-6, wherein the viscoelastic agent is replaced with either a 15% dodecyl ester-HA, sodium salt (approx. 200 kDa) or 43% or 52% benzyl ester-HA, sodium salt (approx. 200 kDa final, modified viscoelastic) were prepared in similar manner to the method disclosed in Example 7. The compositions were incubated at 4° C., RT and 37° C. through 9.5 weeks. The dodecyl or benzyl alcohol (the breakdown products of the respective hydrophobic ester side chains) was quantified using the GC method of Example 10.

After a given incubation time, samples were mixed with four volumes of acetone, which caused precipitation of the polysaccharide. The precipitated polysaccharide was then centrifuged down and the supernatant was analyzed by GC for the presence of the appropriate alcohol.

Full hydrolysis of the dodecyl or benzyl ester-HA side chains would result in 70 ppm dodecyl alcohol or 400 ppm benzyl alcohol in the supernatant, respectively. The results are disclosed in Table 6.

TABLE 6
Percent Hydrolysis of HA-Esters Stored in Phosphate Buffered
Saline at 4° C.

| | Storage Time at | |
|---|---|---|
| Composition | 4° C. | % Hydrolysis |
| 52% benzyl ester-HA | 3.5 weeks | 1.8 |
| 43% benzyl ester-HA | 9 weeks | 5.0 |
| 43% benzyl ester-HA | 9.5 weeks | 12.8 |
| 15% dodecyl ester-HA | 6 weeks | 6.8 |
| 15% dodecyl ester-HA | 9.5 weeks | 12.1 |

As illustrated above, hydrolysis of the comparative modified viscoelastics was greater than 1% through various time points. Because it is desirable for viscoelastic compositions to have storage stability (i.e., viscoelastic products typically require a two year shelf-life expiration date), viscoelastics exhibiting the above described rates of hydrolysis are considered to be less useful in compositions of the present invention.

### EXAMPLE 13

A 3% solution of benzyl ester of HA at 50% carboxylic acid substitution (approximately 200 kDa) was prepared in phosphate buffer with sodium chloride and in citrate/acetate buffer with balanced salts. These solutions formed optically clear, viscoelastic gels which were easily aspirated through a 27 gauge needle. These solutions had low shear viscosities comparable to Viscoat® or Provisc® at 25° C. (k.e., approximately 200 Pa-s) and were shear thinning like Provisc® or Viscoat® at 25° C. These solutions showed a significant drop in viscosity from approximately 200 Pa-s at surgical temperature (25° C.) to 20 Pa-s at body temperature (37° C.).

### EXAMPLE 14

A 1% solution of the dodecyl ester of HA at 14.3% carboxylic acid substitution (approximately 200 kDa) was prepared in citrate/acetate buffer with balanced salts to form a clear, viscoelastic solution. This solution showed a comparable rheological profile to Provisc® or Viscoat®. Viscosity at 25° C. and at shear rates below 0.085/s was approximately 90 Pa-s. Shear thinning began at 0.24/s with a viscosity of 75 Pa-s. At 5.4/s viscosity had-dropped to approximately 16 Pa-s. At 31° C., low shear viscosity was only about 45 Pa-s. At a constant shear stress of 2.89 Pa, the viscosity of this formulation dropped from approximately 100 Pa-s at 25° C. to about 25 Pa-s at 37° C.

### EXAMPLE 15

A 0.75% solution of the dodecyl ester of HA at 14.3% carboxylic acid substitution (approximately 200 kDa) was prepared in citrate/acetate buffer with balanced salts to form a clear, viscoelastic solution. This solution showed a low shear viscosity of about 25 Pa-s at 25° C. and was shear thinning to below 0.1 Pa-s at 534/s. This formulation also showed a decrease in viscosity at constant shear stress (1.1 Pa) from about 25 Pa-s at 25° C. to about 5 Pa-s at 37° C.

### EXAMPLE 16

A 2% solution of the dodecyl ester of HA at 14.3% carboxylic acid substitution (approximately 200 kDa) was prepared in citrate/acetate buffer with balanced salts to form a clear, thick solution. This solution (approximately 2 cc) was autoclaved at 125° C. for about 20 minutes exposure time and was cooled with slow exhaust. After cooling to room temperature, the formulation retained enough viscosity to yield a useful, viscoelastic gel.

### EXAMPLE 17

A 5% solution of the hexadecyl ether of carboxymethylcellulose (containing hexadecyl moieties ether linked to 5% of the repeating monosaccharide units and at approximately 100 kDa) was prepared in phosphate buffers with sodium chloride. The solution was clear and qualitatively formed a viscous gel.

### EXAMPLE 18

A solution of a 1.0 wt % of 20% dodecylamide of HA, sodium salt with 3% chondroitin sulfate and was prepared in PBS by slowly dissolving at 50° C. for two days. Another sample containing only 1.0 wt % of 20% dodecylamide of HA, sodium salt was likewise prepared in PBS by slowly dissolving at 50° C. for two days. After dissolving the samples were each transferred into separate 10-mL syringes and subjected to 100 passes through a dual hub connector to another empty syringe to provide adequate mixing. The samples were centrifuged to remove air bubbles and aspirated through a 27-gauge needle onto the sample plate of a Bohlin CS-10 Constant Stress Rheometer. Rheological analysis was performed to obtain plots of viscosity versus shear rate at 25° C. for both samples. Both samples gave apparent viscosity values of approximately 90 Pa-s in the low shear plateau region of the viscosity versus shear rate plot.

### EXAMPLE 19

The following is a description of the IOP Spike Model.

IOP Spike Model:

The IOP Spike Model employs (1) a perfusion apparatus, pump and pressure transducer/recorder as depicted diagrammatically in FIG. 7; (2) perfusion medium; (3) dissected human eyes; and (4) the viscoelastic material(s) to be tested.

1. Perfusion Medium

The perfusion medium used in the IOP Spike Model is prepared by adding 5 mL of a penicillin-streptomycin solution (10,000 units/mL penicillin (base) from penicillin G and 10,000 μg/mL streptomycin (base) from streptomycin sulfate) and 0.85 mL of a gentamicin solution (10 mg/mL) to 500 mL of a cell culture medium (Dulbecco's modified Eagle's medium, low glucose, with L-alanyl-L-glutamine and pyruvate (Life Technologies, Grand Island, N.Y.)). The perfusion medium is then filtered using a 500 mL sterile filter unit (0.2 μm pore size) and stored at 4° C. (brought to 37° C. before use).

2. Eve Preparation

Cadaver eyes useful in the IOP Spike Model must: i) not be older than 24-36 hours post-mortem when prepared for use in the model; ii) be stored as whole eyes in moist chambers; iii) be devoid of HIV, hepatitis or other infectious agents; and iv) not have undergone ocular surgeries such as glaucoma filtration, scleral buckle implantation or IOL implanation. Anterior segment 7 (see FIG. 10) of a human eye is prepared by the following dissection method:

The eye is carefully trimmed of excess muscle or connective tissue using straight, fine scissors (Katena No. K4-7440), and placed in a container containing a povidone iodine solution (1% free iodine) at 25° C. for approximately 2 minutes. The eye is then removed from the iodine solution, rinsed thoroughly with saline solution, and positioned such that the cornea 3 is centered on top (see FIG. 9a). Referring to FIGS. 9a and 9b, the sclera 5 is then scored (using a sterile ophthalmic crescent knife (Alcon No. 8065-940001) with 24 evenly spaced linear cuts 9 extending radially from the limbus towards the ora serrata (each cut not to exceed 50% depth of the sclera and about 5 mm length) to open the episcleral veins and provide an exit route for the perfusion medium. Referring to FIG. 9b, the globe is then cut into two halves along the horizontal plane 11 approximately midway between the equatorial plane 13 and the scleral plane 15. The anterior (top) half of the eye is separated from the posterior (bottom) half which is discarded. The anterior half is turned over so that the cornea is facing down, and residual vitreous is then carefully removed from the anterior half using Graefe forceps (Katena No. K5-4821). The zonules are then cut with Wescott scissors (Katena No. K4-4100) and the lens removed from the anterior segment using the Graefe forceps. Dressing forceps (Katena No. K5-4010) are then used to remove the iris, and the choroid is circumferentially cut at the ora seratta with the Wescott scissors. Any residual pigment from inside the sclera is then removed with the dressing forceps. Remaining anterior segment 7 is then rinsed two times with perfusion medium to wash out pigment, tissue remnants, or other debris.

3. Perfusion Apparatus

The perfusion apparatus used in the IOP Spike Model is a modified version of that described in the perfusion systems of Johnson and Tschumper and of Clark et al. (Johnson and Tschumper, “Human trabecular meshwork organ culture: a new method,” Invest. Ophthalmol. Vis. Sci., 28:945-953 (1987); and Clarke, et al., “Dexamethasone-Induced Ocular Hypertension in Perfusion-Cultured Human Eyes,” Invest. Ophthalmol. Vis. Sci., 36(2):478-489 (1995)). The critical modifications to the prior systems are the reduction of the chamber volume from about 0.8-1.0 mL to about 0.5-0.6 mL to more nearly approximate the volume of the pseudophakic human anterior segment volume, and the inversion of the chamber during perfusion. The volume reduction is achieved by a protruding island on the platform of the chamber which reduces the space within the dome of the anterior segment and should prevent or reduce stagnation of the viscoelastic solution in the posterior dead space, i.e. posterior to the trabecular meshwork. Turning the chamber upside down (relative to the prior systems) during perfusion is believed to prevent stagnation of the viscoelastic from occurring at the corneal concavity, as such viscoelastic should be effectively mixed by the perfusand which comes out of the elevated island in the direction of that concavity.

Perfusion apparatus 1 is illustrated in FIGS. 7, 8 and 10. Apparatus 1 is comprised of base 2, cylinder 4, island 6, o-ring 8, a plurality of screws 10 and cap 12. In use, apparatus 1 also comprises anterior segment 7.

Base 2 is disk-shaped having top 22, bottom 24 and side 26, and containing channels 14 and 16, platform 18 and threads 19, shaped and sized, to receive screws 10. Channel 14 communicates with opening 28 of side 26 and opening 20 of island 6. Channel 16 communicates with opening 30 of side 26 and opening 32 of platform 18. Channels 14 and 16 are shaped and sized in order to provide for the precise flow (channel 14) and accurate pressure measurement (channel 16) of perfusion medium, to and from apparatus 1. Opening 28 is sized and shaped to receive a fitting (to be connected to infusion line 29) and opening 30 also is sized and shaped to receive a fitting (to be connected to transducer line 31). The fittings are standard connectors known in the art to be useful for receiving tubing or other cylindrical lines. Platform 18 protrudes from base 2, has side 34 and is conically shaped and sized to receive anterior segment 7. Island 6 protrudes from the center of platform 18.

Referring to FIGS. 8 and 10, cylinder 4 is coaxially and permanently situated on top 22 of base 2, and extends flush therefrom. Island 6 has opening 20, contains a portion of channel 14 and extends from platform 18. O-ring 8 has annular, concave interior edge 36 and a plurality of holes 38 sized and shaped for receiving through o-ring 8, screws 10. Edge 36 is sized and shaped such that, when apparatus 1 is put in use, the compression of o-ring 8 against base 2 sandwiches the periphery of anterior segment 7 between edge 36 and side 34 of platform 18, forming anterior chamber 42. As described above, the volume of anterior chamber 42 has been designed by the inventors to approximate the combined volumes of anterior chamber and crystallin lens of a human eye (generally about 0.5-0.6 mL).

As shown in FIGS. 8 and 10, cap 12 is shaped and sized to receive a portion of cylinder 4 and forming closed space 40. The preferred perfusion apparatus for the IOP Spike Model will employ a polysulfone base 2, a polysulfone island 6, a polysulfone o-ring 8, nylon screws 10, a transparent polysulfone cylinder 4, medical steel channels 14 and 16, and a transparent polystyrene cap 12.

4. Assembly and Preparation of Perfusion Apparatus:

Prior to use, apparatus 1 is disassembled and the individual parts are autoclaved or cold sterilized and then soaked in a laminar flow chamber with a sporicidin disinfecting solution (50 ml/L water) followed by an overnight rinse in sterile deionized water.

Referring to FIGS. 7 and 10, perfusion medium is fed to pump, which in turn is connected to infusion line 29, which will infuse perfusion medium through channel 14 and into chamber 42; and transducer line 31 is connected to calibrated pressure transducer and recorder capable of recording the pressure of chamber 42. Apparatus 1 is then reassembled by first connecting fittings disposed at openings 28 and 30 to the infusion and transducer lines, respectively. Apparatus 1 is then arranged with top 22 facing up. Anterior segment 7 is placed on platform 18, cornea side up. Slight perfusion medium flow is then applied via syringe through channel 16 in order to properly seat segment 7 on platform 18. O-ring 8 (which is approximately 1.5 inches in diameter, outer circumference and 0.710-0.736 inches in inner diameter) is then placed over segment 7. It is important that o-ring 8 seats well with segment 7 in order to avoid leaks (a slightly different diameter o-ring 8 may be used in order to improve the seat). Screws 10 are inserted through holes 38 and into threads 19 and tightened evenly to ensure that o-ring 8 is evenly seated. As o-ring 8 is tightened onto the periphery of segment 7, the flow applied through channel 16 should be relieved, such that excessive pressure is not applied to segment 7 upon seating. Screws 10 are torqued against base 2 such that the periphery of segment 7 is tightly sandwiched between platform 18 and o-ring 8, but not so tightly that segment 7 is ruptured. Using syringes, perfusion medium is then pushed through channel 14 while simultaneously pulling perfusion medium out channel 16 through opening 30, slanting base 2 such that any bubbles present will flow out of chamber 42 via channel 16. After the bubbles have been purged, channels 14 and 16 are closed to perfusion medium flow. Apparatus 1 is then returned to level with top 22 facing up.

Cap 12 is then placed over cylinder 4, thereby forming space 40. Apparatus 1 is then inverted so that bottom 24 is facing up, and placed in a tissue culture incubator (Nuaire) maintaining humidified atmosphere (5% CO2/95% air) at 37° C. Transducer line 31 and infusion line 29 should be positioned against the seal of the incubator door, so as not to be damaged or crimped when the door is closed. Pressure transducer should be kept level with apparatus 1. In this configuration apparatus 1 is now ready to be used in the IOP Spike Model.

5. Initial Perfusion:

The perfusion line is opened and the pump operated (setting “6” for Harvard Model No. 944) so that perfusion medium flows freely through opening 28 and channel 14. The pump is allowed to run until the pressure rises to about 5-10 mm Hg, the pump s speed is then decreased to about 2 μL/min (setting “12”) thereafter. Segment 7 is perfused for up to about 24 hours prior to injection of the viscoelastic candidate. If a stable baseline at a pressure of between 10-40 mm Hg is not established within 24 hours, the flow rate can be adjusted repeatedly for a perfusion volume of 2-3 ml each time until a stable baseline IOP is achieved. If the problem is not resolved for another 24 hours, and subsequent flow rate adjustment and flushing steps have not remedied the problem within 48 hours, anterior segment 7 should be considered unreliable and the perfusion terminated.

6. Viscoelastic Injection and Perfusion:

Once perfused anterior segment 7 has generated a stable baseline IOP between 10-40 mm Hg for a period of no less than 4 hours (perfusion rate at between about 1.75-2.05 μl/min) it is ready for IOP spike studies. Typically, such a point is reached 24 hours after initial perfusion. The Model is first validated with injection of a “positive”. control. The positive control is 0.5 mL of diluted sodium hyaluronate (approximatley 750 kDal available from Lifecore Biomedical, Inc., Chaska, Minn.), which is prepared by diluting one part of 3.5% HA in buffering solution to nine parts of the same buffering solution, wherein each 1 mL of the buffering solution contains approximately 0.45 mg sodium dihydrogen phosphate hydrate, 2.00 mg disodium hydrogen phosphate, 4.3 mg sodium chloride (with Water For Injection, USP grade, q.s.) and has a neutral pH. The positive control of 0.5 mL diluted HA (0.35%) is injected into a tubing loop of similar volume attached to multi-valve assembly 33 on perfusion line 29, and multi-valve assembly 33 is switched to permit the complete sample volume to be flushed into perfusion apparatus 1. Thus, the rate of injection is determined by the rate of perfusion. IOP is continuously monitored and recorded. Any IOP spike above the baseline IOP is observed and recorded. If the positive control results in an IOP spike of between 20-80 mm Hg above baseline within 24 hours of injection, the Model is considered validated, and may be used to test candidate transitional viscoelastics.

Generally, transitional viscoelastic samples may be injected in a single anterior segment 2 times, at one day intervals (the first injection being that of the positive control). Sample viscoelastics should be diluted to one tenth the original concentration using the same buffering solution used to prepare the control sample. A stable and acceptable baseline IOP should be reached before each new injection, as indicated by the decline of any IOP spike generated by a preceding viscoelastic sample. If the baseline IOP is not regained within the range of 10-40 mm Hg within one day, any further perfusion in a given anterior segment should be discontinued.

As stated above, a transitional viscoelastic of the present invention (i.e. causing little or no IOP spike) will exhibit an average spike of 10 mm Hg or less above baseline.

### EXAMPLE 20

A transitional viscoelastic of the present invention, AL-12488, 43% substituted benzyl ester modified HA (approximately 200 kDal) was tested in the IOP Spike Method described above and compared to the positive control (benchmark) and 200 kDal HA from Fidia. The results of the study are represented graphically in FIG. 11. The arrows indicate the various injections. Injections 1 and 4 were 0.35% positive control HA, injection 2 was 0.35% Fidia unmodified HA, and injection 3 was 0.35% AL-12488. All injection samples were 0.5 ml in volume. The asterisks indicate the individual IOP spikes resulting from the injections. Only the transitional viscoelastic (AL-12488) can be characterized as exhibiting little or no IOP spike, as the spike observed therefor in the IOP Spike Model is not more than, and in fact is considerably less than, about 10 mm Hg above the baseline IOP.

Those skilled in the art will appreciate that the suitability of a given transitional viscoelastic for a particular step in a surgical procedure will depend upon such things as the viscoelastic's concentration, average molecular weight, viscosity, pseudoplasticity, elasticity, rigidity, adherence (coatability), cohesiveness, molecular charge, and osmolality in solution. The viscoelastic's suitability will depend further on the function(s) which the viscoelastic is expected to perform and the surgical technique being employed by the surgeon.

An appropriate buffer system (e.g., sodium phosphate, sodium acetate or sodium borate) may be added to the compositions to prevent pH drift under storage conditions.

Because all or a significant portion of the transitional viscoelastics of the present invention may be left in the eye at the close of surgery, these viscoelastics are uniquely adapted to serve the dual roles of viscosurgical tool and drug delivery device.

Ophthalmic drugs suitable for use in the compositions of the present invention include, but are not limited to: anti-glaucoma agents, such as beta-blockers including timolol, betaxolol, levobetaxolol, carteolol, miotics including pilocarpine, carbonic anhydrase inhibitors, prostaglandins, seratonergics, muscarinics, dopaminergic agonists, adrenergic agonists including apraclonidine and brimonidine; anti-infective agents including quinolones such as ciprofloxacin, and aminoglycosides such as tobramycin and gentamicin; non-steroidal and steroidal anti-inflammatory agents, such as suprofen, diclofenac, ketorolac, rimexolone and tetrahydrocortisol; growth factors, such as EGF; immunosuppressant agents; and anti-allergic agents including olopatadine. The ophthalmic drug may be present in the form of a pharmaceutically acceptable salt, such as timolol maleate, brimonidine tartrate or sodium diclofenac. Compositions of the present invention may also include combinations of ophthalmic drugs, such as combinations of (i) a beta-blocker selected from the group consisting of betaxolol and timolol, and (ii) a prostaglandin selected from the group consisting of latanoprost; 15-keto latanoprost; fluprostenol isopropyl ester (especially 1R-[1α(Z),2β(1E,3R*),3α,5α]-7-[3,5-dihydroxy-2-[3-hydroxy-4-[3-(trifuoromethyl)-phenoxy]-1-butenyl]cyclopentyl]-5-heptenoic acid, 1-methylethyl ester); and isopropyl [2R(1E,3R),3S(4Z),4R]-7-[tetrahydro-2-[4(3-chlorophenoxy)-3-hydroxy-1-butenyl]-4-hydroxy-3-furanyl]-4-heptenoate.

In the event a pharmaceutical agent is added to the transitional viscoelastics, such agents may have limited solubility in water and therefore may require a surfactant or other appropriate co-solvent in the composition. Such co-solvents typically include: polyethoxylated castor oils, Polysorbate 20, 60 and 80; Pluronic® F-68, F-84 and P-103 (BASF Corp., Parsippany, N.J., USA); cyclodextrin; or other agents known to those skilled in the art. Such co-solvents are typically employed at a level of from about 0.01 to 2 wt. %. It may also be desirable to add a pharmaceutically acceptable dye to the viscoelastic to improve visualization of the viscoelastic during surgery and/or to stain ocular tissue (especially the capsular bag during capsulorhexis in cataract surgery) for improved visualization of such tissue. The use of such dyes in conventional viscoelastics is described in WO 99/58160. Preferred dyes include trypan blue, trypan red, brilliant crysyl blue, and indo cyanine green. The concentration of the dye in the viscoelastic solution will preferrably be between about 0.001 and 2 wt. %, and most preferably between about 0.01 and 0.1 wt %. However, it will be appreciated by those skilled in the art that any such additive (pharmaceutical agents, co-solvents, or dyes) may only be employed to the extent that they do not detrimentally affect the viscoelastic properties of the compositions of the present invention.

The methods of the present invention may also involve the use of various viscoelastic agents having different adherent or cohesive properties. Those skilled in the art will recognize that the compositions of the present invention may be employed by the skilled surgeon in a variety of surgical procedures.

Given the advantages of each type of viscoelastic, the surgeon may employ various viscoelastic compositions of the present invention in a single surgical procedure. While the use of the transitional viscoelastic of the present invention have not been previously disclosed for use in surgeries, U.S. Pat. No. 5,273,056 (McLaughlin et al.) discloses methods which exploit the use of compositions employing viscoelastics of varying viscoelastic properties during a given ocular surgery, the entire contents of which are incorporated herein by reference.

For example, for portions of surgical procedures involving phacoemulsification. and/or irrigation/aspiration, e.g., cataract surgery, it is generally preferable to use a viscoelastic agent that possesses relatively greater adherent properties and relatively lesser cohesive properties. Such viscoelastic agents are referred to herein as “adherent” agents. The cohesiveness of a viscoelastic agent in solution is thought to be dependent, at least in part, on the average molecular weight of that agent. At a given concentration, the greater the molecular weight, the greater the cohesiveness. Those portions of surgical procedures involving manipulation of delicate tissue are generally better served by viscoelastic agents that possess relatively greater cohesive properties and relatively lesser adherent properties. Such agents are referred to herein as “cohesive” agents. For cohesive agents such as these, which are being employed primarily for tissue manipulation or maintenance purposes as opposed to protective purposes, a functionally desirable viscosity will be a viscosity sufficient to permit the skilled surgeon to use such agent as a soft tool to manipulate or support the tissue of concern during the surgical step(s) being performed.

For other viscoelastic agents, which are being employed primarily for protective purposes (“adherent” agents) as opposed to tissue manipulation purposes, a functionally desirable viscosity will be a viscosity sufficient to permit a protective layer of such agent to remain on the tissue or cells of concern during the surgical step(s) being performed. Such viscosity will typically be from about 3,000 cps to about 60,000 cps (at shear rate of 2 sec⁻¹ and 25° C.), and preferably will be about 40,000 cps. Such adherent agents are capable of providing the protective function previously discussed, yet are not prone to inadvertent removal, which could jeopardize the delicate tissue being protected. Unfortunately, this same characteristic makes aspiration of such adherent viscoelastics at the end of surgery (as recommended for all such commercially available products in cataract surgery) problematic for surgeons, and may result in the coated tissues being subjected to trauma during the removal procedure. A significant advantage of the transitional viscoelastics of the present invention is that they may be left in the surgical site at the close of surgery thereby avoiding unnecessary trauma to the affected soft tissues.

Preferred methods of the present invention will employ the use of multiple viscoelastics in a given surgical procedure, wherein at least one of such viscoelastics is a transitional viscoelastic. In a most preferred embodiment of the invention, a transitional viscoelastic possessing superior adherent properties is used in cataract surgery, at the close of which some or all of the transitional viscoelastic is left in situ and causes little or no IOP spike.

The invention has been described by reference to certain preferred embodiments; however, it should be understood that it may be embodied in other specific forms or variations thereof without departing from its spirit or essential characteristics. The embodiments described above are therefore considered to be illustrative in all respects and not restrictive, the scope of the invention being indicated by the appended claims rather than by the foregoing description.

1. A sterile, non-inflammatory viscoelastic composition comprising a polymeric agent in an aqueous solution, wherein the composition exhibits a zero shear viscosity of at least 1 Pa-s at 25° C., and wherein the viscoelastic composition exhibits little or no IOP spike when tested in a validated IOP Spike Model.

2. The composition of claim 1, wherein the polymeric agent is selected from the group consisting of: hydrophobically modified polysaccharides or mucopolysaccharides (with or without surfactants); dialyzed polyampholytes or dialyzed mixtures of oppositely charged polyelectrolytes; mixtures of polysaccharides or mucopolysaccharides with cationic hydrophilic polymers; polysaccharides and hydrophilic synthetic polymers with temperature dependent conformational transitions; and combinations thereof.

3. The composition of claim 2, wherein the solution exhibits a zero shear viscosity at 25° C. of from about 5 to about 10,000 Pa-s, and a Viscosity Factor of about 1,000 to about 20,000.

4. The composition of claim 3, wherein the polymeric agent is a hydrophobically modified polysaccharide selected from the group consisting of HA-amides, HA-esters, HA-amines, HA-ethers, HA-thioethers, HA-alkyls and combinations thereof.

5. The composition of claim 4, wherein the polysaccharide is an HA-amide selected from the group consisting of octylamide HA, dodecylamide HA, and hexadecylamide HA.

6-14. (canceled)

15. A sterile, non-inflammatory transitional viscoelastic composition comprising a hydrophobically modified polysaccharide in an aqueous solution, wherein the solution exhibits a zero shear viscosity of at least 1 Pa-s at 25° C., and wherein the zero shear viscosity of the solution decreases by at least 50% when the solution undergoes a temperature change from about 25° C. to about 37° C.

16. The composition of claim 15, wherein the solution exhibits a zero shear viscosity at 25° C. of from about 5 to about 10,000 Pa-s, and a Viscosity Factor of about 1,000 to about 20,000.

17. The composition of claim 16, wherein the hydrophobically modified polysaccharide is selected from the group consisting of HA-amides, HA-esters, HA-amines, HA-ethers, HA-thioethers, and HA-alkyls and mixtures thereof.

18. The composition of claim 17, wherein the polysaccharide is an HA-amide selected from the group consisting of octylamide HA, dodecylamide HA, and hexadecylamide HA.

19-26. (canceled)
